(19) United States
(12) Patent Application Publication
(10) Pub. No.: US 2026/0060551 A1
(43) Pub. Date: Mar. 5, 2026

(54) METHOD AND SYSTEM FOR HIGHLY PARALLEL ULTRASONIC DETECTION USING MICRO-RING RESONATOR IN AN ARRAY FORMAT

(71) Applicant: Northwestern University, Evanston, IL (US)

(72) Inventors: Youngseop LEE, Evanston, IL (US); Roman V. KURANOV, Evanston, IL (US); Hao ZHANG, Evanston, IL (US); Cheng SUN, Evanston, IL (US)

(21) Appl. No.: 19/311,921

(22) Filed: Aug. 27, 2025

Related U.S. Application Data
(60) Provisional application No. 63/688,176, filed on Aug. 28, 2024.

(51) Int. Cl. A61B 5/00; G01N 29/24

(52) U.S. Cl. CPC A61B 5/0095; G01N 29/2418

(57) ABSTRACT

An ultrasonic detection system for a photoacoustic imaging system includes a driving light source and an ultrasonic detector. The ultrasonic detector includes a micro-ring resonator array configured to receive light emitted by the driving light source. The micro-ring resonator array includes a plurality of micro-ring resonators, each respective micro-ring resonator having a respective radius that corresponds to a driving wavelength of the respective micro-ring resonator. The respective radius of each respective micro-ring resonator is different than the respective radii of the other micro-ring resonators. The ultrasonic detection system further includes a photodetector configured to convert optical signals output through the micro-ring resonator array into electrical signals and processing circuitry configured to receive the electrical signals and reconstruct two-dimensional and/or three-dimensional images of a sample based thereupon. Methods for photoacoustic imaging include using the ultrasonic detection system to detect ultrasonic pressure waves via intensity- or phase-based detection.

### CROSS-REFERENCE TO RELATED APPLICATIONS

This application claims the benefit of priority to U.S. Provisional Patent Application No. 63/688,176, filed Aug. 28, 2024, which is hereby incorporated by reference in its entirety.

### STATEMENT REGARDING FEDERALLY SPONSORED RESEARCH OR DEVELOPMENT

This invention was made with government support under grant numbers GM143397 and GM135018 awarded by the National Institutes of Health. The government has certain rights in the invention.

### BACKGROUND OF INVENTION

Photoacoustic (PA) imaging enables noninvasive volumetric imaging of biological tissues by detecting the endogenous optical absorption contrast. Since the scattering of ultrasonic waves is two orders of magnitude lower than the optical scattering, PA imaging is better suited for deep tissue imaging. Optical resolution PA microscopy (OR-PAM) makes it possible to image deeper into the tissue in comparison to confocal microscopy at a given optical irradiation wavelength thanks to the significantly reduced scattering of the ultrasonic waves. However, the attainable imaging depth is still constraint by the optical scattering of the incident focused illumination. PA computed tomography (PACT) mitigates this issue by exploiting the diffusive light to illuminate the deep into the tissues, delivering ultrasonically defined spatial resolution at depths far beyond the optical diffusion regime around 1 mm, which is beyond the reach of conventional optical imaging modalities.

### SUMMARY OF THE INVENTION

Provided herein are systems for optical-based ultrasound detection using multiple detectors being arranged in either one-dimensional (1D) or two-dimensional (2D) format, as well as corresponding methods for optical-based ultrasound detection. Specifically, the present disclosure provides systems and methods that utilize micro-ring resonators (MRRs) as ultrasound detection elements, and in particular, that utilize 1D arrays or 2D arrays of MRR-based ultrasound detectors. The systems and methods are suitable for parallel detection and reconstruction of an ultrasonic wavefront using a rapid wavelength sweeping light source. The present disclosure further provides methods for nano-fabrication of 2D arrays of MRR-based ultrasound detectors.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1B: A block diagram of photoacoustic (PA) signal detection and PA imaging by using micro-ring resonator (MRR) arrays with a tunable light source (FIG. 1A), and a schematic illustration of photoacoustic signal generation from a sample and photoacoustic signal detection by using MRR arrays with the tunable light source (FIG. 1B).

FIG. 2: A working principle of the MRR arrays with the tunable source for intensity-based detection of PA signals. The time-varying intensity modulations induced by PA signals are recorded, and their temporal evolution is aggregated to reconstruct the corresponding PA signal in the time domain.

FIG. 3: Signal acquisition method using an internal timeclock from the tunable source as a triggering signal.

FIG. 4: A working principle of the MRR arrays with the tunable source for phase-based detection of PA signals.

FIG. 5: A working principle of the MRR arrays with the tunable source for balanced detection of PA signals using a balanced detector. A balanced photodetector is employed to enhance signal fidelity by suppressing common-mode noise during the detection of PA-induced optical modulations and thus, enhancing the signal-to-noise ratio (SNR) in PA signal detection. Balanced detection can be applied for both intensity-based and phase-based detection.

FIGS. 6A-6B: Various types of MRR arrays for on-chip ultrasound detection. FIG. 6A: various configurations of MRR arranged in 1D or 2D array connected to a single pair of swept source and detector. This configuration can be expanded by using one or more swept sources and detectors. FIG. 6B: alternative strategy of expanding the MRR arrays by adding optical delay lines so fewer swept sources and detectors are needed.

FIGS. 7A-7B: Nanofabrication results of 1D array format of polymer MRR.

FIGS. 8A-8E: Ultrasonic signals measured from each of 5 MRRs. FIG. 8A: time domain of the ultrasonic signal acquired from an MRR (Ring #1) showing output (V) as a function of time (t) (top panel) and frequency domain of the ultrasonic signal measured from an MMR (Ring #1) showing amplitude (a.u.) as a function of frequency (MHz) (bottom panel). FIG. 8B: time domain of the ultrasonic signal acquired from an MRR (Ring #2) showing output (V) as a function of time (t) (top panel) and frequency domain of the ultrasonic signal measured from an MMR (Ring #2) showing amplitude (a.u.) as a function of frequency (MHz) (bottom panel). FIG. 8C: time domain of the ultrasonic signal acquired from an MRR (Ring #3) showing output (V) as a function of time (t) (top panel) and frequency domain of the ultrasonic signal measured from an MMR (Ring #3) showing amplitude (a.u.) as a function of frequency (MHz) (bottom panel). FIG. 8D: time domain of the ultrasonic signal acquired from an MRR (Ring #4) showing output (V) as a function of time (t) (top panel) and frequency domain of the ultrasonic signal measured from an MMR (Ring #4) showing amplitude (a.u.) as a function of frequency (MHz) (bottom panel). FIG. 8E: time domain of the ultrasonic signal acquired from an MRR (Ring #5) showing output (V) as a function of time (t) (top panel) and frequency domain of the ultrasonic signal measured from an MMR (Ring #5) showing amplitude (a.u.) as a function of frequency (MHz) (bottom panel).

FIGS. 9A-9D: Parallel ultrasonic signal detection using 2 MRRs by sweeping the driving wavelength of the swept source.

FIGS. 10A-10E: Parallel ultrasonic signal detection using 5 MRRs by sweeping the driving wavelength of the swept source.

FIGS. 11A-11C: Scanning Electron Microscope (SEM) images of nanofabrication results of 2D array format. FIG. 11A: Si Template. FIG. 11B: PDMS. FIG. 11C: PS MMR.

FIG. 12: SEM images of enlarged view of each polymer MRR in a 2D array.

FIG. 13A provides a SEM image of 1 by 5 MRR array with drop lines. The transmission spectrum of the MMR array was measured with drop lines by using the tunable laser and photodetector through each output drop line #1-#5 and a total output port. FIG. 13B provides a SEM image of enlarged view of one MMR unit comprising of a bus waveguide for input and output, a ring resonator, and a drop line. FIG. 13C provides a measured resonance spectrum from the total output port and drop lines. The result shows resonance wavelengths in total spectrum match the output through the drop lines.

FIG. 14A provides a SEM image of first, third, and fifth MRR of 1 by 5 MRR array with an initial radius of 40 μm and an incremental radius of 18 nm. FIG. 14B provides a measured resonance spectrum of the 1 by 5 MRR array. The results show clearly resolvable five resonance curves in one free spectral range (FSR). The average Q-factor is 1.1×10⁴.

FIG. 15 provides a SEM image of 3 by 3 MRR array with one bus waveguide.

FIG. 16A provides a SEM image of first, fourth, and seventh ring resonator of 3 by 3 MRR array with an initial radius of 40 μm and an incremental radius of 12 nm.

FIG. 16B provides measured resonance spectrum of the 3 by 3 MRR array. The result shows the clearly resolvable 9 resonance curves in one free spectral range (FSR). Their average Q-factor is 1.4×10⁴.

FIG. 17 provides measured ultrasonic signals from each ring of the 3 by 3 MRR array. The input ultrasonic signal has a center frequency of 5 MHz.

FIG. 18A provides a schematic illustration of parallel detection of ultrasonic signal using the MRR array. FIG. 18B provides the reconstructed ultrasonic signal from each ring of the 1 by 5 MRR array in parallel detection. The input ultrasonic signal has a center frequency of 5 MHz. The wavelength sweeping frequency is 9.1 MHz. FIG. 18C provides the reconstructed ultrasonic signal from each ring of the 3 by 3 MRR array in parallel detection. The input ultrasonic signal has a center frequency of 5 MHz. The wavelength sweeping frequency is 5.3 MHz.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

As used herein, the term “tunable source” refers to a radiation and/or light emitting device, such as a laser or other optical emitter, configured to generate output at a selectable or variable wavelength, frequency, or other emission parameter over a defined range. The emission characteristics of the tunable source may be adjusted by mechanical, electrical, thermal, or optical means, enabling spectral tuning in response to control signals or environmental conditions. An example of a tunable source includes a swept source.

As used herein, the term “swept source” refers to type of tunable source that is a radiation-emitting device, for example a laser, configured to emit light whose output wavelength varies continuously or discretely over time across a defined spectral range. The wavelength sweep may be achieved through electrical, mechanical, thermal, or optical tuning mechanisms. Examples of swept sources include tunable external cavity lasers, distributed feedback (DFB) lasers with tunable elements, micro-electromechanical systems (MEMS)-tunable vertical-cavity surface-emitting lasers (VCSELs), and Fourier domain mode-locked (FDML) lasers.

In the context of the present disclosure, a “sample” refers to any material, composition, or object capable of generating detectable photoacoustic waves upon absorption of electromagnetic radiation. Such photoacoustic waves are produced by a thermoelastic expansion of light-absorbing constituents within the sample, which then propagate as acoustic signals. The sample may possess inherent optical absorbing properties or may be rendered optically absorptive through the introduction of exogenous contrast agents. The sample may be static or dynamic, uniform or heterogeneous, and of any physical state (e.g., solid, liquid, gaseous, or a combination thereof), provided it permits the propagation of both the excitation electromagnetic radiation and the generated photoacoustic waves to and from a detection region. A sample may be biological, chemical, synthetic, or naturally occurring. The sample may include internal or external structures of interest to be imaged, characterized, or monitored using photoacoustic techniques.

The performance of the PACT is critically relying on the characteristic performance of the ultrasound detectors being used. An array of ultrasound detector is highly desirable for rapidly recording ultrasonic wavefront needed for PACT reconstruction. Furthermore, an ideal ultrasound detector requires high detection sensitivity over a broad range of ultrasound frequencies to maximize the axial resolution, and a diffraction-limited detector with the lateral dimension comparable with the corresponding ultrasound wavelength to achieve the optimal resolution and the maximum field of view (FOV).

Typical commercial ultrasonic detection methods rely on piezoelectric transducers. However, these have the technical limitations: 1) limited imaging resolutions due to narrow frequency bandwidth; 2) relative low detection sensitivity in the broad frequency range; 3) limited field-of-view due to narrow acceptance angle; 4) high fabrication cost for realizing the array format; 5) huge system complexity of the array format; and 6) fabrication limitation in 2D array. Miniaturizing the sensing area of such piezoelectric transducers will unfavorably increase their impedance and thus, compromise their sensitivity, thereby imposing challenges in further developing ultrasonic detectors into a two-dimensional (2D) array format. Due to the aforementioned limitations, the commonly used piezoelectric detectors fail to fulfill the requirements desired for the performance of PACT.

The present disclosure provides novel systems and methods for optical-based ultrasound detection via the use of detectors arranged in either one-dimensional (1D) or two-dimensional (2D) format. Specifically, the present disclosure provides systems and methods that utilize micro-ring resonators (MRRs) as ultrasound detection elements. The present disclosure further provides methods for nano-fabrication of 1D or 2D arrays of MRR-based ultrasound detectors, as well as systems and methods for parallel detection of an ultrasonic wavefront using a rapid wavelength sweeping light source, and for detection and reconstruction of the ultrasonic wavefront.

The present disclosure recognizes that polymer MRRs based optical ultrasonic detection can overcome the limitations of the traditional/commercial piezoelectric ultrasonic transducers. The MRR can provide wide bandwidth of frequency, high detection sensitivity in the broad frequency range, and wide field-of-view. In addition, the array format of polymer MRR array can be fabricated by low-cost and reproducible nano-fabrication methods based on soft nanoimprinting lithography. Furthermore, using a tunable source as a driving light source can provide highly parallel ultrasonic detection through the MRR array based ultrasonic detector array.

The systems and methods of the present disclosure provide for novel ultrasonic detection with high detection sensitivity, broad frequency range, and a wide acceptance angle (field of view). The systems and methods of the present disclosure improve photoacoustic imaging speed and quality (e.g. resolution) via the array format of ultrasonic detectors. Furthermore, the systems and methods of the present disclosure can be easily integrated with other imaging modalities (e.g. due to small form factor and optical transparency). In addition, methods of the present disclosure provide for low-cost and reproducible fabrication of ultrasonic detectors.

In various embodiments, systems and methods of the present disclosure provide the following:
1. Fast and highly sensitive ultrasound detection with MRR arrays.
2. The ability to using one tunable laser by fast wavelength hopping/sweeping to drive one or more MRR.
3. Intensity-based detection.
4. Phase-based detection.
5. The configuration for balanced detection.
6. Signal acquisition of MRR arrays using internal timeclock of the tunable laser.
7. On-chip ultrasound detector.

According to a first aspect, the present disclosure provides systems and methods for MRR array based ultrasonic detection with a tunable light source. Specifically, fast and highly sensitive ultrasonic detector systems include MRRs arrays and a single tunable laser source for faster volumetric photoacoustic (PA) imaging with a high resolution. The MRR arrays comprise 1 by n, n by 1, or n by n micro-rings with different radius, which facilitates tailored resonance wavelengths.

According to a second aspect, the tunable laser source, such as a swept source, can provide short hopping time in few nanoseconds and a narrow pulse bandwidth less than few picometers, which enables acquisition of data at least two points in a period of PA signals through the MRR arrays. This system, and a corresponding method for using this system, allows the use of single tunable laser source to drive multiple MRRs, for fast and highly sensitive ultrasound detection.

According to a third aspect, the present disclosure provides systems and methods for intensity-based detection. The intensity-based detection employs the variations in the intensity through MRR arrays for given excitation wavelengths due to the resonance shifts of MRRs induced by the ultrasonic pressure waves from samples. Each excitation wavelength of the tunable light source corresponds to each resonance wavelength of MRR arrays. In the intensity-based detection, the detection sensitivity is maximized at the maximum slope of the resonance curve. In addition, using a balanced detector can reduce noises of PA signals additionally.

According to a fourth aspect, the present disclosure provides systems and methods for phase-based detection. The phase-based detection method measures the changes in the phase shift through MRR arrays using additional couplers. The phase of the light is shifted after the light travels through the ring from 0 to 2π depending on the wavelength and the optical path length. The phase shift of the MRR can be measured by using an additional waveguide coupled to the original bus waveguide before and after the MRR based on interference.

According to a fifth aspect, the present disclosure provides systems and methods for implementing balanced detection. A balanced photodetector is employed to enhance signal fidelity by suppressing common-mode noise during the detection of PA-induced optical modulations and thus, enhancing the signal-to-noise ratio (SNR) in PA signal detection. Balanced detection can be applied for both intensity-based and phase-based detection.

According to a sixth aspect, the present disclosure provides systems and methods that utilize a unique signal acquisition technique that employs an internal timeclock of the tunable laser source as a triggering signal. Signals through the MRR array can be recorded by sweeping the wavelength of the tunable laser from an off-resonance wavelength to a driving wavelength of each MRR. Then, a PA signal from each ring of the MRR array can be extracted and reconstructed from a whole recorded signal data by using an off-resonance signal as a triggering signal.

According to a seventh aspect, the present disclosure provides methods for fabricating on-chip ultrasound detectors from various semiconductors and polymer materials, and further provides systems including on-chip ultrasound detectors fabricated by such methods. MRR arrays and waveguide couplers can be fabricated on a single chip with a compact size (<mm²˜cm²) by using conventional nanolithography methods. Depending on the working wavelengths, various materials such as silicon (Si), silicon nitride (SiNx), silicon dioxide (SiO₂), polystyrene, SU-8, etc. can be used for waveguide core materials.

Embodiments of the present disclosure can replace traditional ultrasound detection based on piezoelectric materials in photoacoustic imaging, or broadly ultrasound imaging applications. Using a single driving light source with a swept laser instead of multiple narrow band lasers can reduce the total system cost. Furthermore, an array format of micro-ring resonators can be fabricated with low-cost and high reproducibility. Embodiments of the present disclosure thereby facilitate widespread usage of ultrasonic imaging in biomedical imaging applications.

According to an embodiment, an ultrasonic detector for a photoacoustic imaging system is provided. The ultrasonic detector includes a MRR array configured to receive light emitted by a driving light source. The MRR array includes a plurality of micro-ring resonators, each respective MRR having a respective radius that corresponds to a driving wavelength of the respective MRR. The respective radius of each respective MRR is different than the respective radii of the other MRRs.

In variations of the ultrasonic detector, each MRR is configured to deform in the presence of ultrasonic pressure waves emitted from a sample.

In variations of the ultrasonic detector, it further includes a bus waveguide that connects each respective MRR of the plurality of MRR in series. In variations, the ultrasonic detector further includes a plurality of bus waveguides, each respective bus waveguide of the plurality of bus waveguides connecting, in series, a respective subset of MRR of the plurality of micro-ring resonators, the plurality of subsets of MRRs being connected in parallel. In variations, at least one respective bus waveguide of the plurality of bus waveguides includes an optical delay line.

According to an embodiment, an ultrasonic detection system for a photoacoustic imaging system includes the ultrasonic detector and a driving light source.

In variations of the ultrasonic detection system, the driving light source is a tunable light source or a broadband laser source comprising a wavelength filter. In variations of the ultrasonic detection system, the driving light source is a swept source laser configured to output a laser beam having a wavelength that varies across a range of wavelengths as a function of time. In variations, the range of wavelengths extends from a first wavelength to a second wavelength. The first wavelength is at or below a wavelength that corresponds to a shortest driving wavelength of the plurality of MRRs, and the second wavelength is at or above a longest driving wavelength of the plurality of MRRs.

In variations, the swept laser source is configured to vary the wavelength of the laser beam between respective driving wavelengths of the plurality of MRRs, wherein the wavelength of the laser beam is varied such that it occupies one or more off-resonance wavelengths between different respective driving wavelengths. In variations, the swept laser source is a pulsed laser source configured to emit a series of pulses, wherein every other pulse has a wavelength that corresponds to a driving wavelength of the plurality of MRRs. In variations, the swept laser source is configured to emit, between pulses that correspond to a driving wavelength of the plurality of MRRs, a pulse having a wavelength that is equal to one or more off-resonance wavelengths.

In variations of the ultrasonic detection system, the system further includes a photodetector configured to convert optical signals output through the MRR array into electrical signals and processing circuitry configured to receive the electrical signals and reconstruct two-dimensional and/or three-dimensional images of a sample. In variations, the processing circuitry is configured to use electrical signals corresponding to optical signals output by the MRR array at points in time at which the wavelength of the laser beam occupies one of the one or more off-resonance wavelengths as a clock signal.

In variations, the optical signals output through the MRR array are modulated by photoacoustic signals emitted by a sample, and the photodetector converts the modulation in the optical signals into the electrical signals.

In variations, the optical signals output through the MRR are a time-sequence of pulsed optical signals that exhibit a time-sequence of modulation with alternating high and low optical power, the photodetector converts the time-sequence of pulsed optical signals into the electrical signals, and the electrical signals are time-modulated pulsed electric signals.

In variations, the time-modulated pulsed electric signals are used as an internal clock, by the processing circuitry, to accurately assign a component of the electrical signals to a deformation of a corresponding ring resonator waveguide of the MRR array. This provides a viable and affordable control scheme that is insensitive to the possible time jittering among the laser wavelength sweeping and transmitted signal, which may compromise the accuracy in assigning the recorded electric signal to the right MRR. Errors in assignment can completely ruin the accuracy of the acoustic signal reconstruction.

In variations, the processing circuitry is further configured to detect phase variations in the optical signals output through the MRR array, wherein the phase variations result from deformations caused by the presence of ultrasonic pressure waves emitted from a sample.

In variations of the ultrasonic detection system, the photodetector comprises a first photodetector port configured to receive the optical signals output by the MRR array, and the ultrasonic detector further comprises a coupler waveguide configured to receive the light emitted by the driving light source and to guide the light emitted by the driving light source to a second photodetector port.

In variations, the photodetector is a balanced photodetector including the second photodetector port, the photodetector being further configured to output second electrical signals, or alternatively, the ultrasonic detector further comprises a second photodetector comprising the second photodetector port, the second photodetector being configured to output second electrical signals, wherein the second electrical signals correspond to second optical signal provided to the second photodetector port. The balanced photodetector may be useful for common-noise rejection. In variations, the balanced photodetector may comprise electronic circuitry that is coupled to an output of the first and second photodetector ports. The electronic circuitry may be configured to produce a single differential output signal that represents the difference between the first and second electrical signals and/or first and second optical signals. Such a configuration may allow for common noise subtraction among two photodetector inputs thereby increasing the signal-to-noise ratio of the ultrasound detection and thus, enhance the detection sensitivity.

In variations, the ultrasonic detection system further includes processing circuitry configured to receive the electrical signals and the second electrical signals and to detect variations in the respective radius of each respective MRR resulting from deformations caused by the presence of ultrasonic pressure waves emitted from the sample.

According to an embodiment, a photoacoustic imaging system includes a pulsed laser configured to illuminate a sample with laser pulses and the ultrasonic detection system. The ultrasonic detector is configured to detect ultrasonic pressure waves emitted from the sample.

According to an embodiment, a method for photoacoustic imaging of a sample includes providing the ultrasonic detector, detecting, by the ultrasonic detector, ultrasonic pressure waves emitted from a sample, and processing, by processing circuitry, electrical signals corresponding to the optical signals output through the micro-ring resonator array to reconstruct a two-dimensional and/or a three-dimensional image of the sample.

In variations of the method, the ultrasonic detector is configured to detect the ultrasonic pressure waves via intensity-based detection. In alternative variations of the method, the ultrasonic detector is configured to detect the ultrasonic pressure waves via phase-based detection.

The invention can be further understood by the following non-limiting examples.

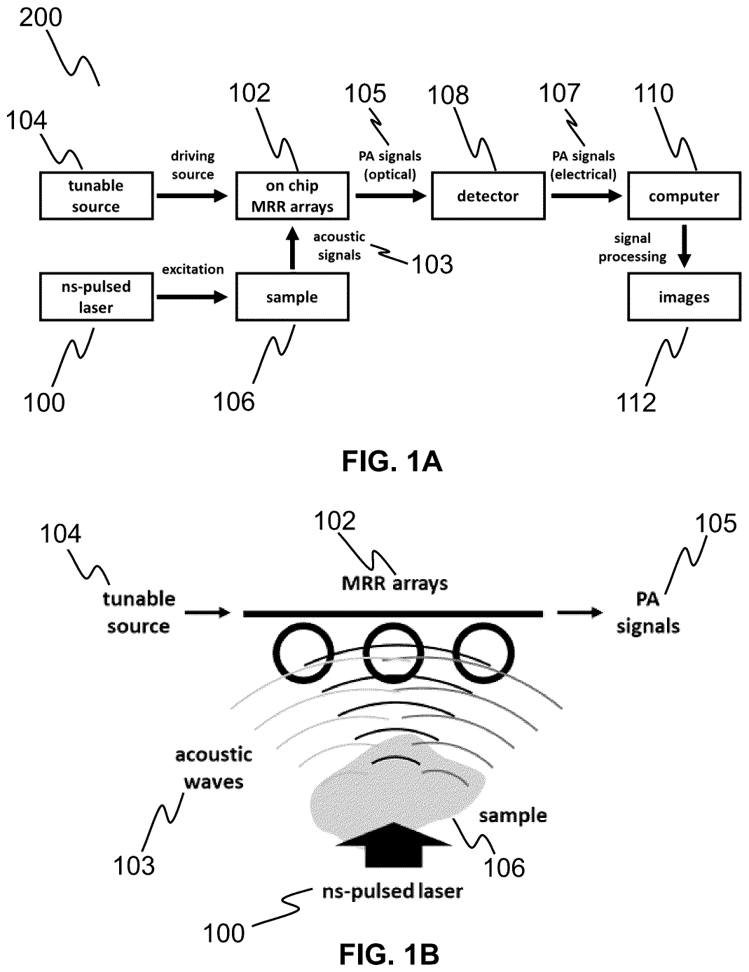

FIG. 1A illustrates a block diagram of a photoacoustic (PA) signal detection and PA imaging system (200) that includes an on-chip micro-ring resonator (MRR) array (102) and a tunable light source (104). On-chip MRR arrays (102) driven by the tunable light source (104) detect PA signals (103) from a sample (106) excited by a nanosecond pulsed laser (100). A photodetector (108) converts optical PA signals (105) to electrical PA signals (107). The two-dimensional or three-dimensional PA images can be reconstructed from the PA signals by post signal processing, for example, with a computer (110) to generate images (112). FIG. 1B provides schematic illustration of photoacoustic signal (103) generation from a sample (106) excited by a nanosecond pulsed laser (100) and photoacoustic signal detection (105) by using MRR arrays (102) with the tunable source (104).

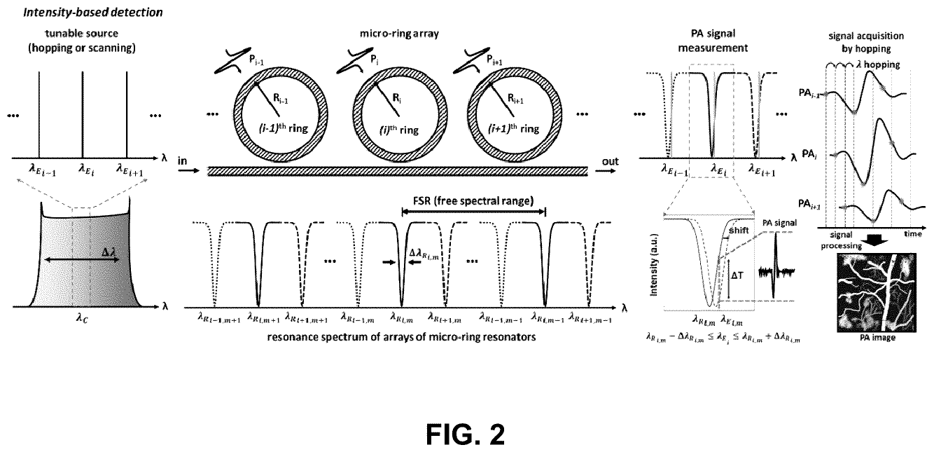

FIG. 2 illustrates a working principle of the MRR array with the tunable source for intensity-based detection of PA signals. MRR arrays with different radius values (Rᵢ, i=1, 2, 3, . . . ) have different resonance wavelengths (λ_{R}_{_{i,m}}, i=1, 2, 3, . . . , m−1, 2, 3, . . . ) and free spectra range (FSR) defined by

$$\frac{{λ}^{2}}{{n}_{g} 2 π {R}_{i}} .$$

Ultrasonic pressure waves from the sample excited by a nanosecond pulsed laser induce the deformation of the MRRs, which causes changes in both the geometry and refractive index of the material of MRRs and subsequently alters the corresponding effective refractive index of the waveguide guided mode. The changes in the optical path length of the MRRs are accumulated while the light wave is circulating inside the rings. The amplified optical path length changes can then be quantified by measuring the wavelength shift of the resonance modes. The resonance shift can be measured as the modulation of the transmitted optical intensity at the given wavelength by using a laser source with a narrow bandwidth. Using the tunable laser source such as a swept laser source with a narrow pulse bandwidth less than few pico-meters and short hopping time in few nanoseconds can measure ultrasonic pressure waves with MRR arrays. Each excitation wavelength of the tunable light source (λ_{E}_{ᵢ}) corresponds to each resonance wavelength of MRR arrays. Acquisition of data at least two points in a period of PA signal (PAᵢ) by hopping the excitation wavelength and post signal processing realize PA image reconstruction by using MRR arrays and the tunable light source. In FIG. 2 and elsewhere, the following variables are defined:
λ_{C}: center wavelength of tunable light source
Δλ: tunable range of tunable light source
λ_{E}_{ᵢ}: excitation wavelength of tunable source for i-th MRR, i=1, 2, 3 . . .
Rᵢ: radius of i-th MRR, i=1, 2, 3 . . .
Pᵢ: detected ultrasonic pressure (or photoacoustic pressure) by i-th MRR, i=1, 2, 3 . . .
A_{R}_{_{i,m}}: resonance wavelength of i-th ring resonator for integer m, m=1, 2, 3 . . .

$${λ}_{{R}_{i , m}} = \frac{{n}_{eff} ( 2 π {R}_{i} )}{m} ,$$

where n_{eff} is an effective index, m=1, 2, 3 . . .
Δλ_{R}_{_{i,m}}: full width at half maximum (FWHM) or bandwidth of a resonance peak of
i-th ring resonator for integer m, m=1, 2, 3 . . .
PAᵢ: PA signal from i-th MRR, i=1, 2, 3 . . .

The wavelength bandwidth (or pulse width) of the MRR driving laser may be narrower than the full width at half maximum (FWHM) of the resonance curve of the MRR. For example, with a high Q-factor value, such as 2×10⁵, a corresponding FWHM may be 3.9 pico-meter at 780 nano-meter. For a low Q-factor value such as 1×10³, a corresponding FWHM may be 780 pico-meter at 780 nano-meter. Therefore, the pulse width of the MRR driving laser may be less than pico-meter level at least. In addition, the narrower bandwidth, such as, for example atto-meter level, can improve detection sensitivity to probe the wavelength shift of the MRRs. For example, a tunable narrowband laser, TLB-6712 (New Focus), has a wavelength bandwidth of 406 atto-meter at a wavelength of 780 nano-meter. In certain applications, the bandwidth may be less than a few pico-meters. In embodiments, therefore, the wavelength bandwidth (or pulse width) may be optionally between 1 atto-meter and 1 nano-meter. Optionally, the wavelength bandwidth may be from approximately 1 pico-meter to 1 nano-meter. Optionally, the wavelength bandwidth may be from approximately 1 atto-meter to 1 pico-meter. In certain embodiments, optionally, the wavelength bandwidth may be less than 500 pico-meters. Optionally, in an embodiment, the wavelength bandwidth may be less than 400 pico-meters. Optionally, in an embodiment, the wavelength bandwidth may be less than 300 picometers, optionally, less than to 250 pico-meters, optionally, less than 200 pico-meters, optionally, less than 100 pico-meters, optionally, less than 50 pico-meters, optionally, less than 10 pico-meters, or optionally, less than 1 pico-meter.

The hopping time or transition time (time between one driving wavelength to next driving wavelength) may be short as possible, such as, for example, a few nano-seconds to minimize transition between driving wavelengths and maximize the duration time that the driving laser activates the MRRs. Optionally, the transition time of the swept laser may be between 1 nano-second to 3 nano-seconds. Optionally, in an embodiment, the hopping time, or transition time of a swept laser may be less than or equal to 10 nano-seconds.

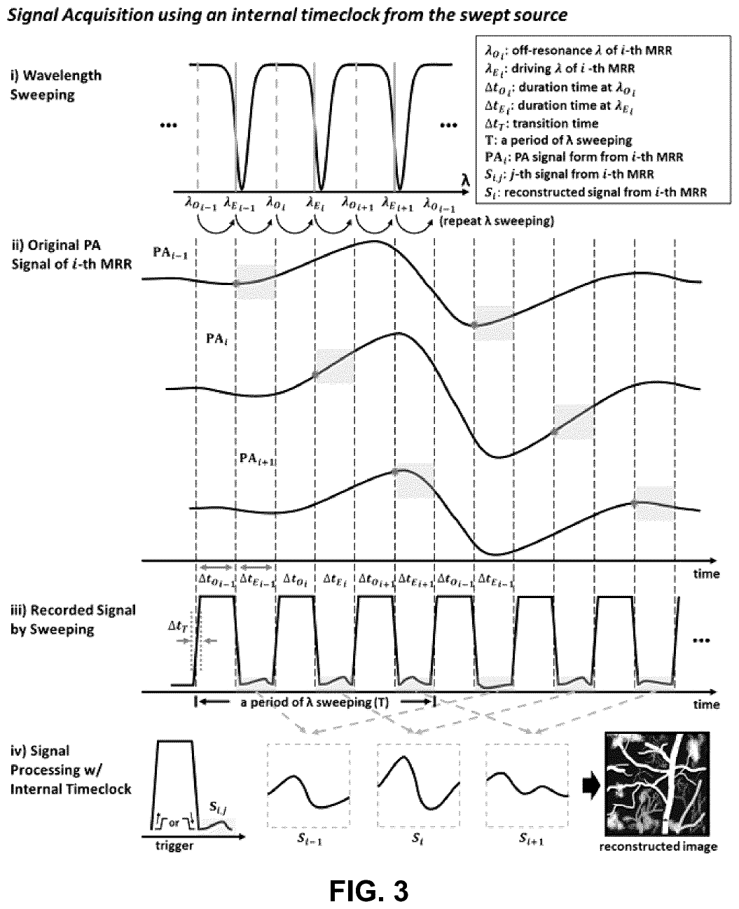

FIG. 3 illustrates a signal acquisition method using an internal timeclock from the swept source as a triggering signal. A wavelength of the driving laser source is rapidly swept from off-resonance wavelength (λ_{O}_{ᵢ}) and driving wavelength (λ_{E}_{ᵢ}) with a duration time, Δt_{O}_{ᵢ} and Δt_{E}_{ᵢ}, for each i-th MRR, where i is an integer. For example, one wavelength sweeping cycle comprises of λ_{O}_{_{i−1}}, λ_{E}_{_{i−1}}, λ_{O}_{ᵢ}, λ_{E}_{ᵢ}, λ_{O}_{ᵢ₊₁}, and λ_{E}_{ᵢ₊₁} in that order. When the wavelength is at λ_{O}_{ᵢ}, the non-resonance signal is measured in the recorded signal. When the wavelength is at λ_{E}_{ᵢ}, a part of PA signal from i-th MRR can be included in a recorded signal. A PA signal from each MRR may be extracted and reconstructed from the whole recorded signal data by using the off-resonance signal at Ao, as a triggering signal (rising edge or falling edge type). Finally, a PA image may be reconstructed using PA signals.

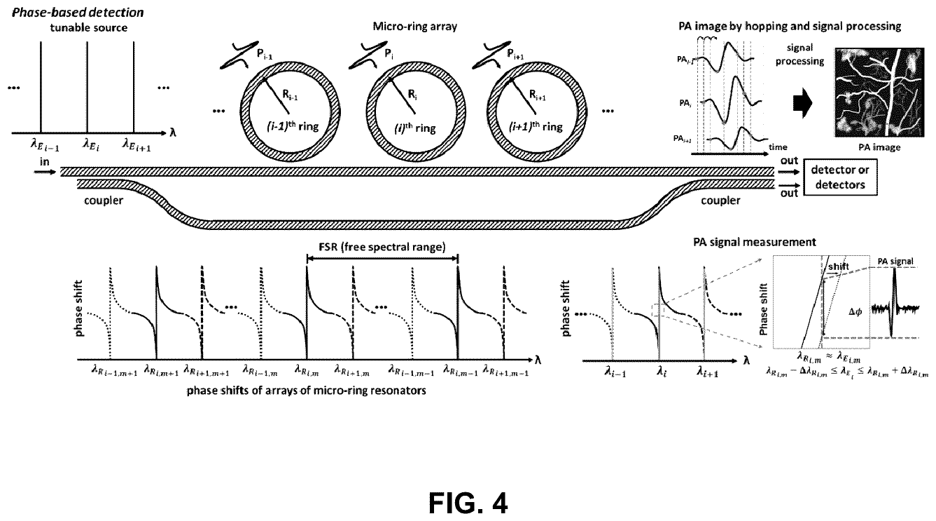

FIG. 4 illustrates a working principle of the MRR arrays with the tunable source for phase-based detection of PA signals. Two output ports may be connected to a single detector or a balanced detector. Phase-based detection uses variation in phase shift of the MRR due to ultrasonic pressure waves. Changes in the optical path length induced by the deformation of the MRR due to ultrasonic pressure waves cause changes in resonance properties of the MRR. Resulting changes in the resonance of the MRR includes variations in the intensity as well as the phase shift of the for the given wavelength. The phase of the light is shifted after the light travels through the ring from 0 to 2π depending on the wavelength and the optical path length. The phase shift of the MRR may be measured by using an additional waveguide coupled to the original bus waveguide before and after the MRR based on interference.

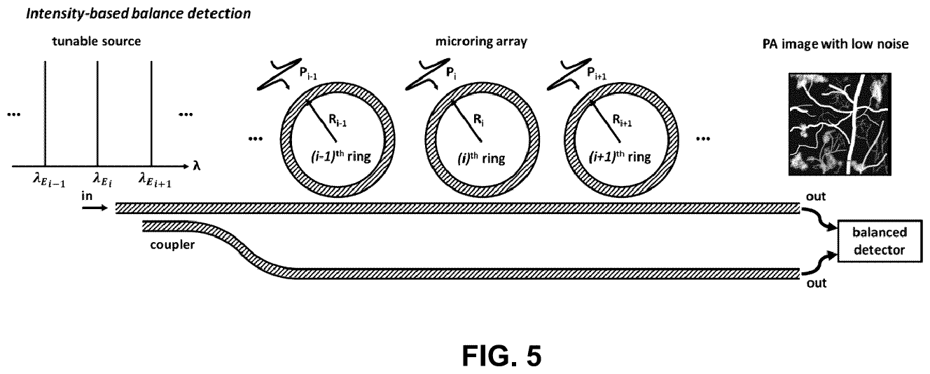

FIG. 5 illustrates a working principle of the MRR arrays with the tunable source for intensity-based detection of PA signals using a balanced detector. Both the original input signals from the tunable source and the PA signals through the MRRs are both detected by using a balanced detector with an optical coupler before the MRRs.

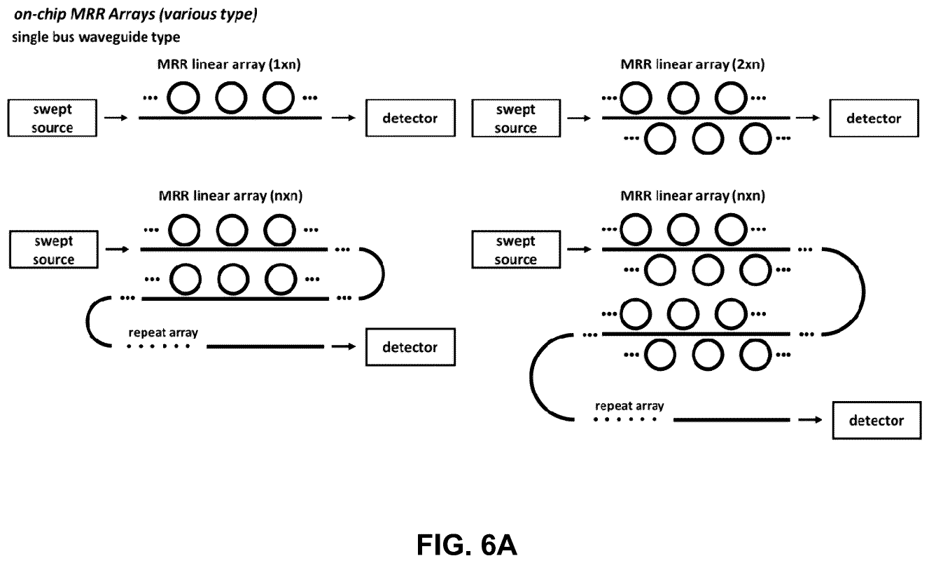

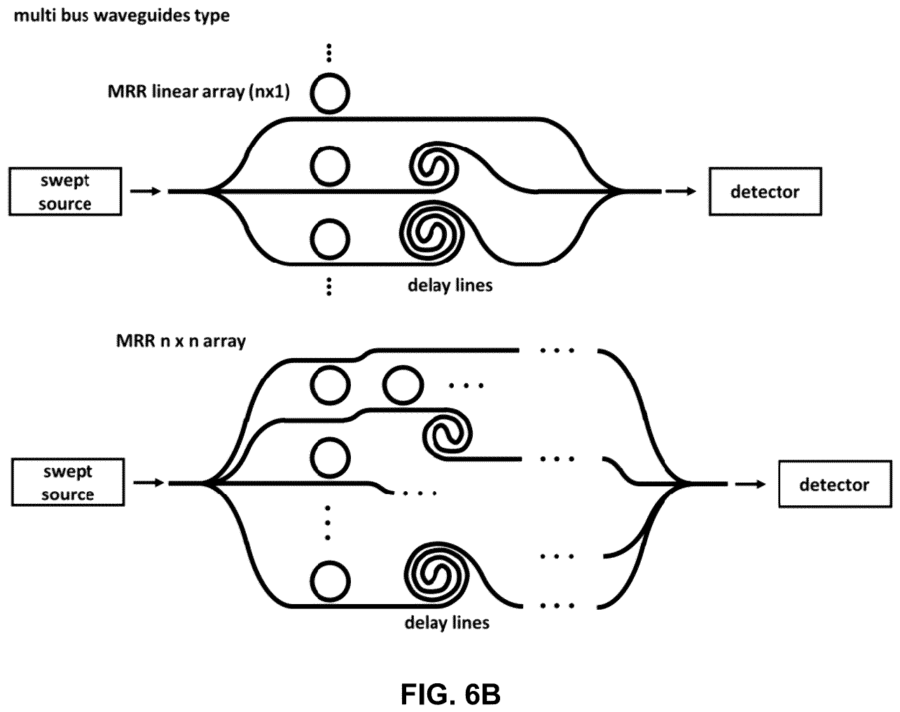

FIGS. 6A-6B illustrate various types of MRR arrays for on-chip ultrasound detection. Reference waveguides for phase-based detection and balance detection types are omitted.

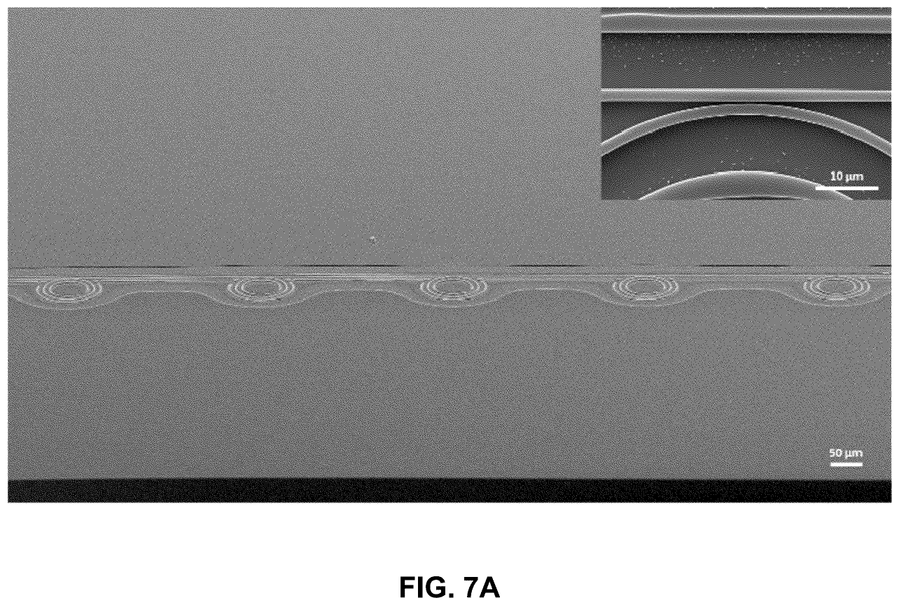

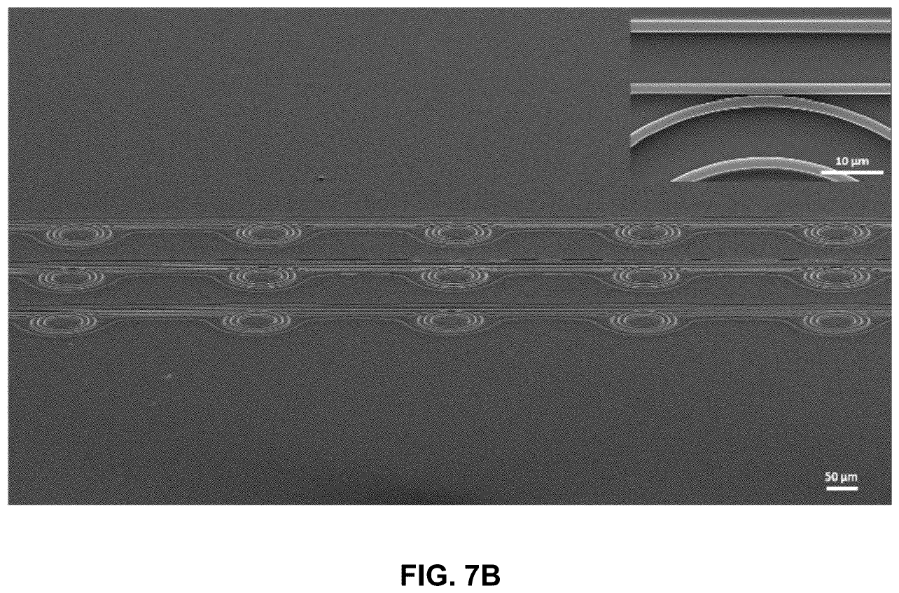

FIGS. 7A-7B illustrate nanofabrication results of 1D array format of polymer MRR. FIG. 7A: A scanning electron microscope (SEM) image of the 1D MRR array with 5 ring resonators. Each row has 5 ring resonators with 5 different radii with an incremental value of 18 nm, which corresponds to a resonance wavelength incremental of ˜0.6 nm. The radius of first, second, third, fourth, and fifth ring is 40 μm, 40.018 μm, 40.036 μm, 40.054 μm, and 40.072 μm, respectively. An inset is an enlarged SEM image of the coupling region of the ring and bus waveguides. FIG. 7B: A SEM image of the three sets of 1D MRR array with 5 ring resonators. Each row set has a different gap distance between the ring resonator and the bus waveguide. The gap value for the first, second, and third row is 300 nm, 250 nm, and 200 nm, respectively. An inset is an enlarged SEM image of the coupling region of the ring and bus waveguides.

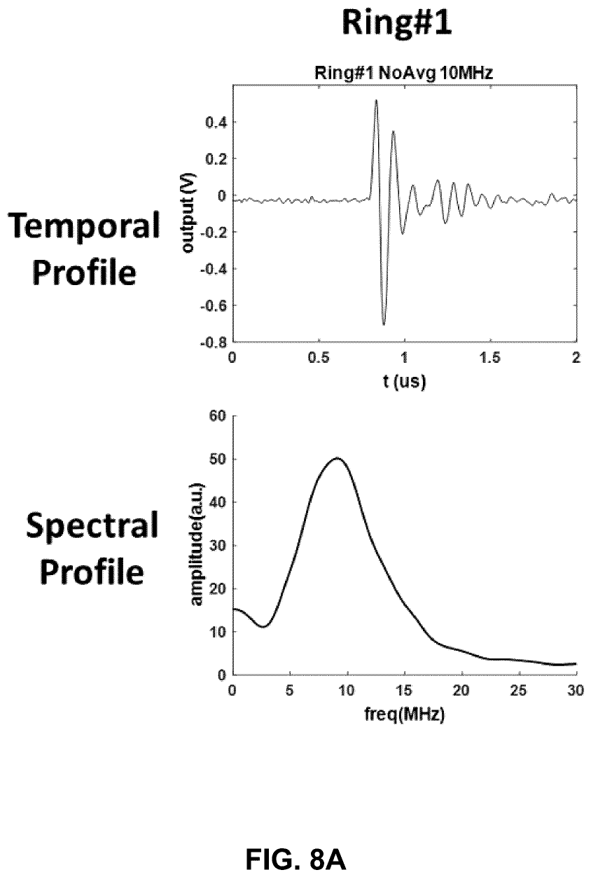

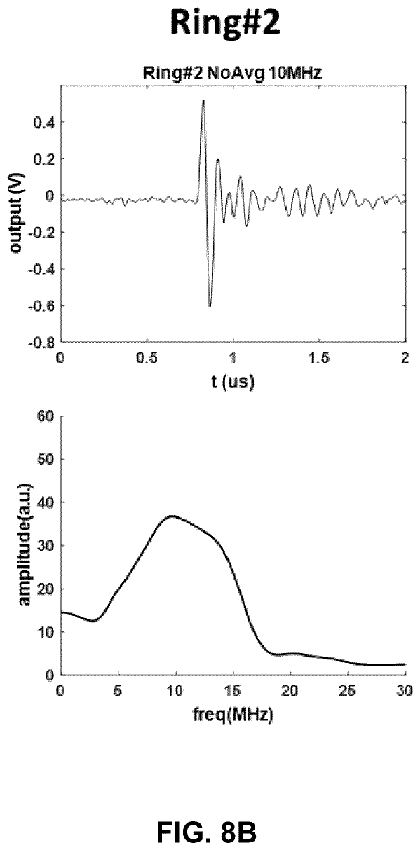

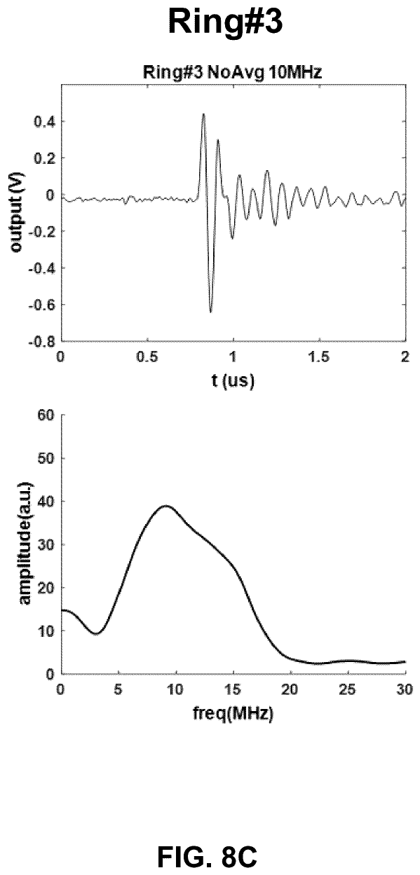

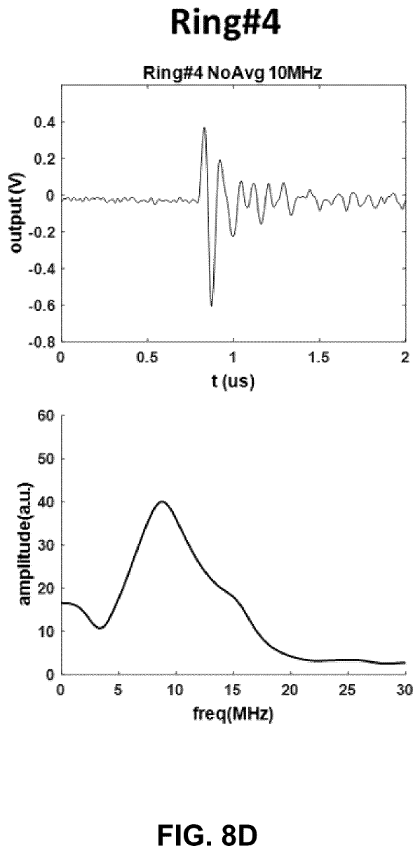

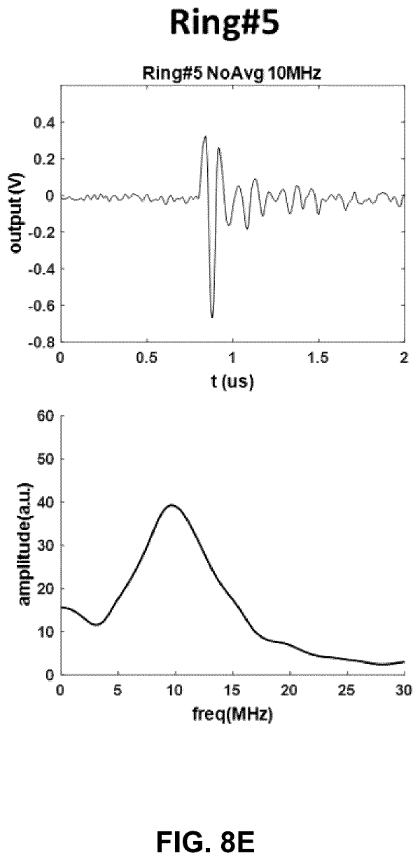

FIGS. 8A-8E illustrate ultrasonic signals with a center frequency of 10 MHz measured ultrasonic signals with a center frequency of 10 MHz from each 5 MRRs.

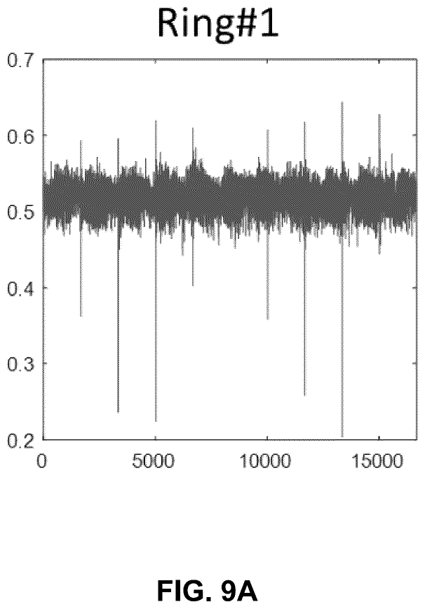

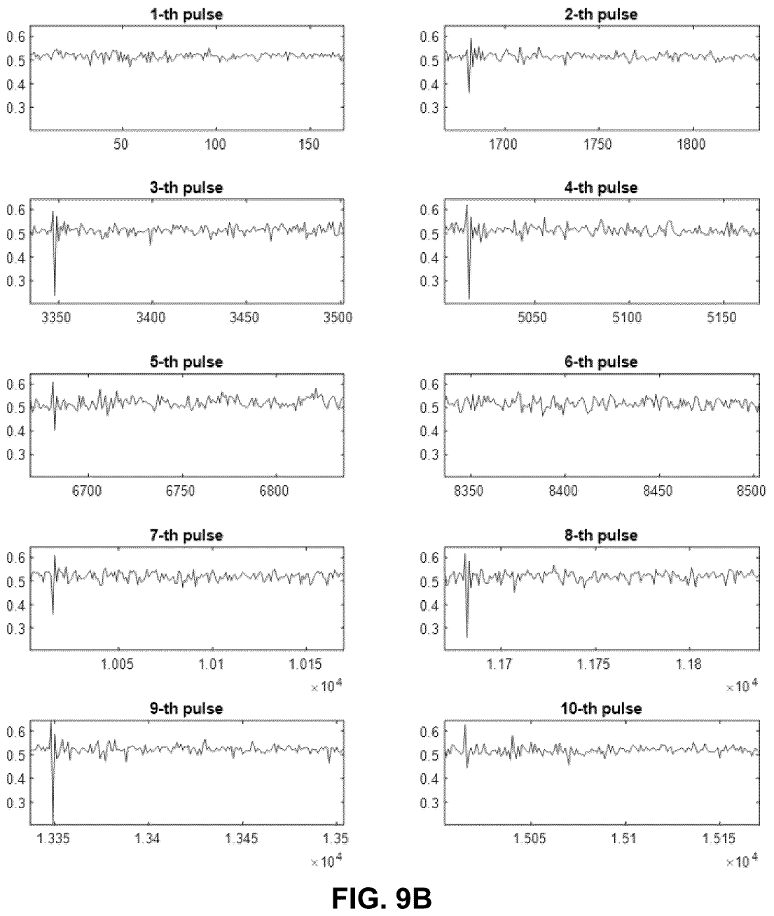

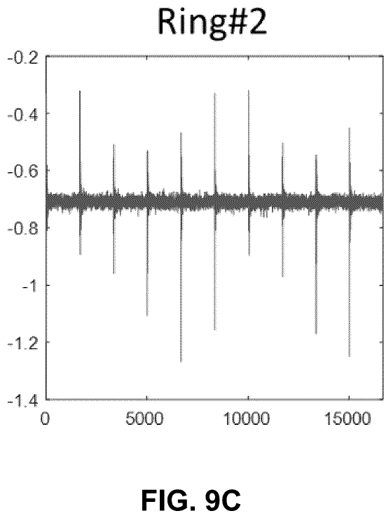

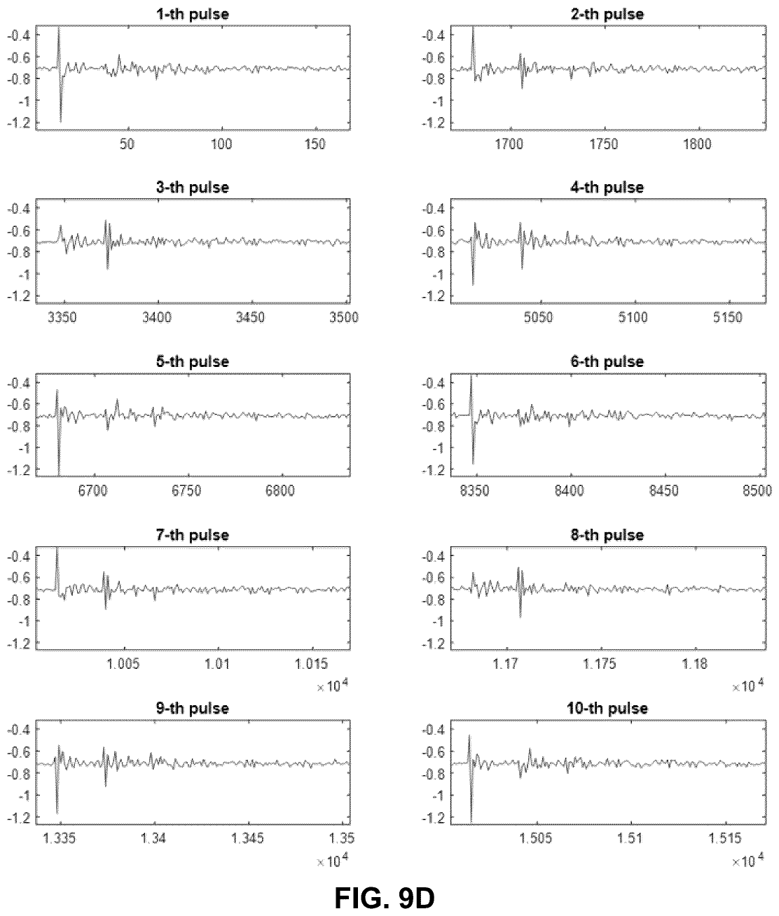

FIGS. 9A-9D illustrate parallel ultrasonic signal detection using 2 micro-ring resonators by sweeping the driving wavelength of the swept source. The input ultrasonic signal has a center frequency of 10 MHz and a repetition rate of 10 kHz. The original signal is segmented by the method described in FIG. 3 and then the segmented signals from the on-state for each ring are averaged and merged (FIGS. 9A and 9C). Segmented and averaged signals clearly show reconstructed ultrasonic signals corresponding to each ultrasonic pulse (FIGS. 9B and 9D).

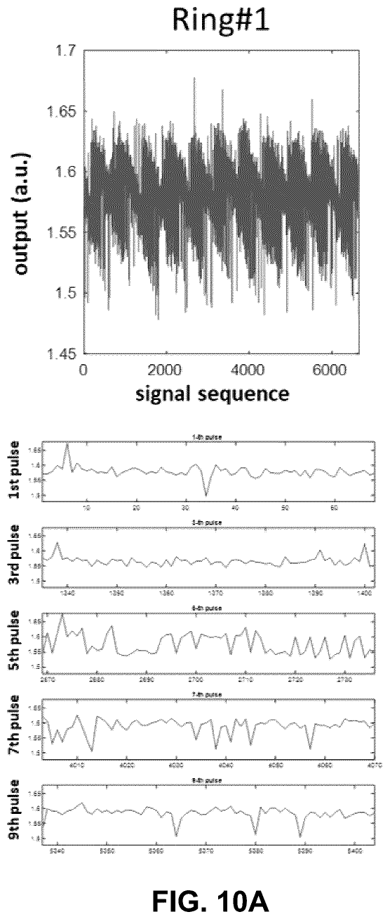

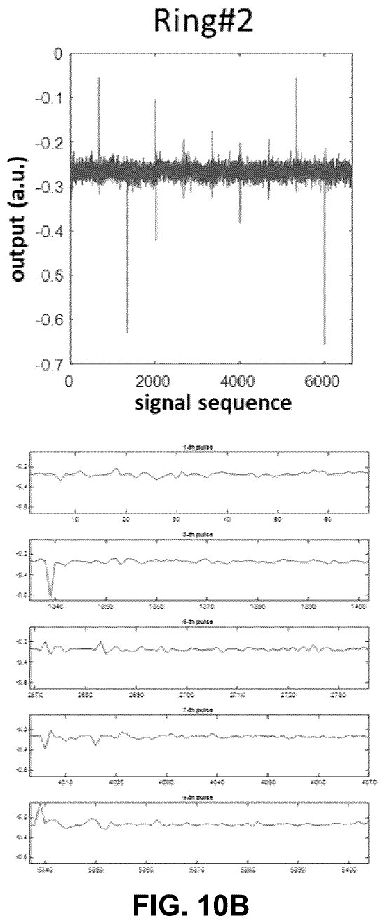

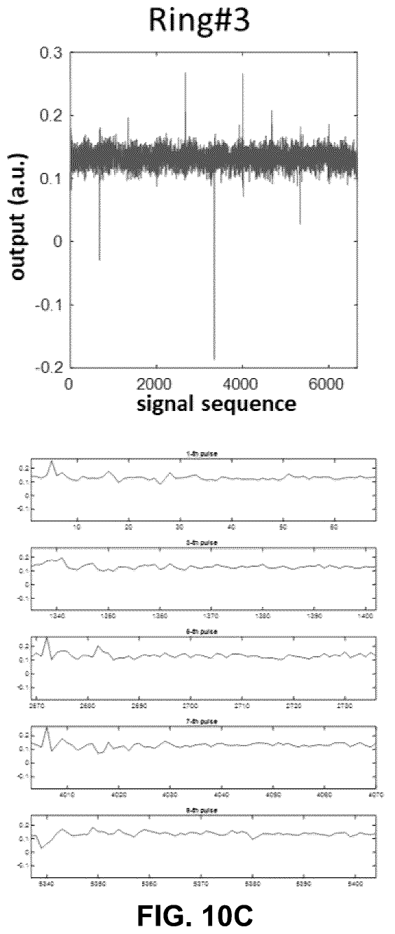

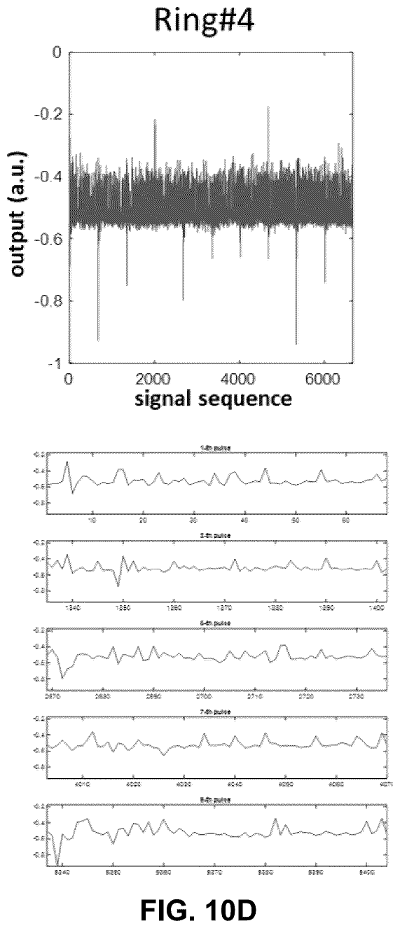

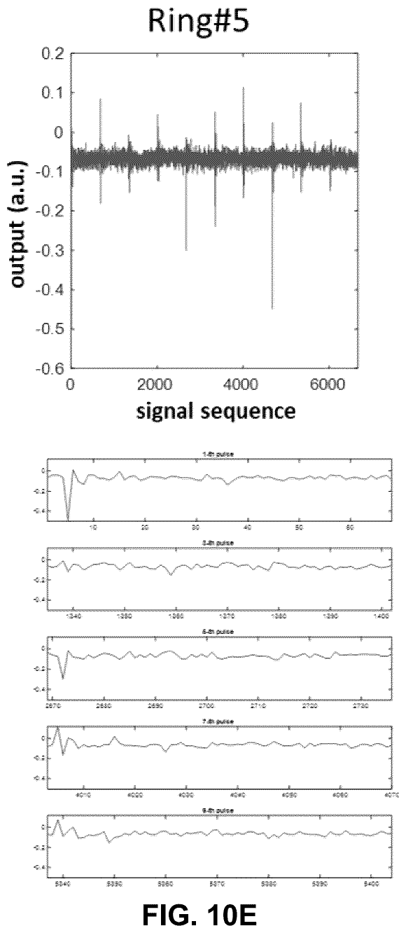

FIGS. 10A-10E illustrate parallel ultrasonic signal detection using 5 micro-ring resonators by sweeping the driving wavelength of the swept source. The input ultrasonic signal has a center frequency of 10 MHz and a repetition rate of 10 kHz. The original signal is segmented by the method described in FIG. 3 and then the segmented signals from the on-state for each ring are averaged and merged (top panel). Segmented and averaged signals clearly show reconstructed ultrasonic signals corresponding to each ultrasonic pulse (bottom).

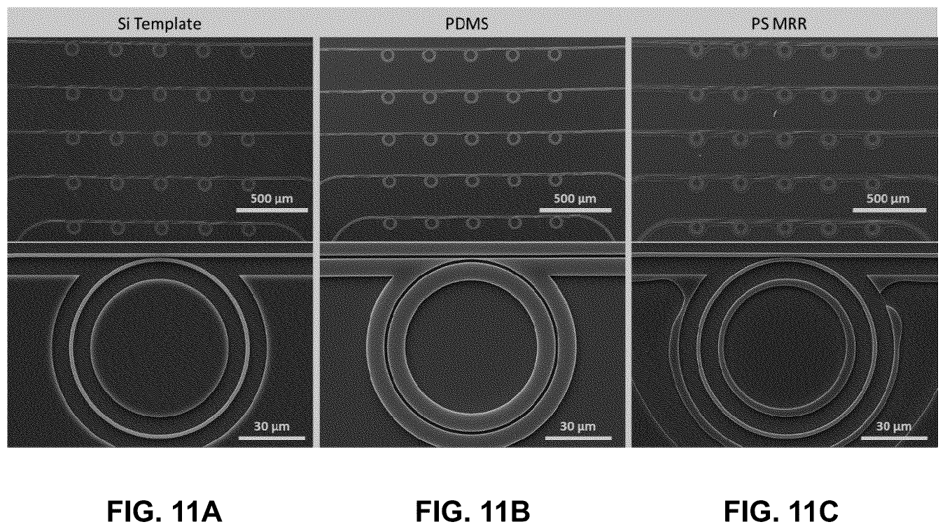

FIGS. 11A-11C provide SEM images of nanofabrication results of 2D array format (5 by 5) of the (FIG. 11A) Si master template, the (FIG. 11B) PDMS mold, and the (FIG. 11C) polymer MRR. Each row has 5 ring resonators with 5 different radii with an incremental value of 18 nm, which corresponds to a resonance wavelength incremental of ˜0.6 nm. The radius of first, second, third, fourth, and fifth ring is 40 μm, 40.018 μm, 40.036 μm, 40.054 μm, and 40.072 μm, respectively. Five rows have the same ring resonator design values.

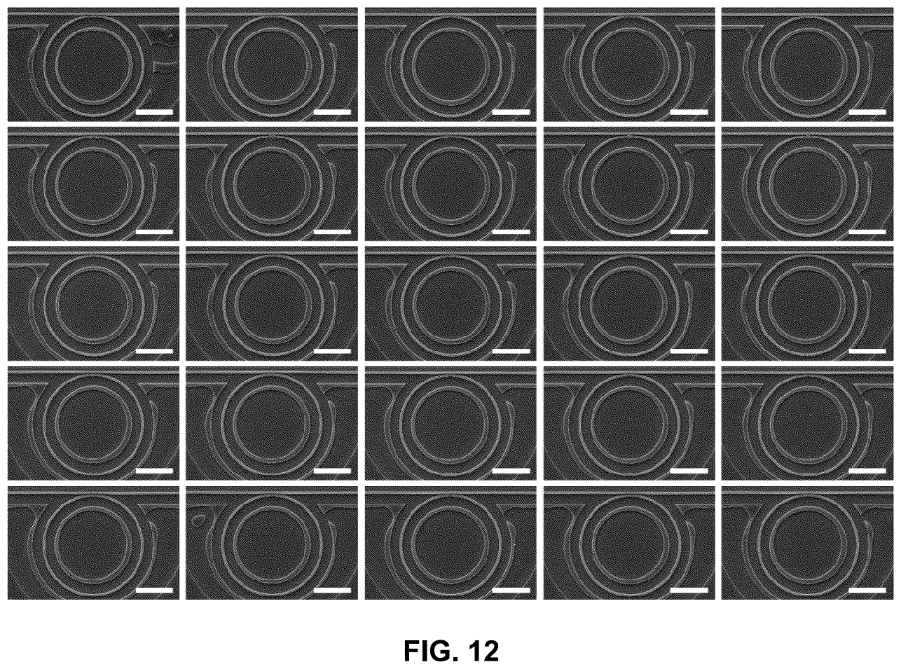

FIG. 12 provides SEM images of enlarged view of each ring resonator in polymer MRR 2D array. All 25 micro-rings are successfully fabricated without defects. The scale bar is 30 μm.

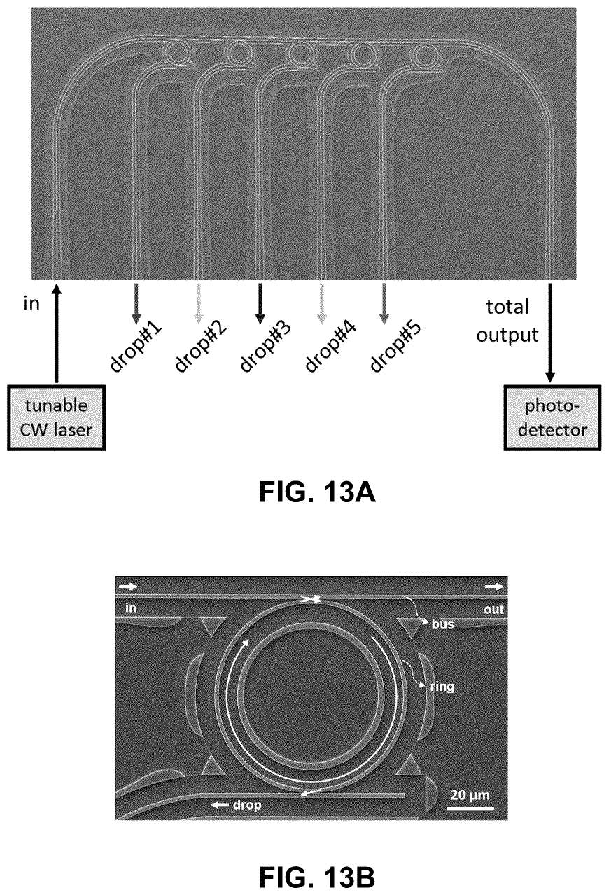

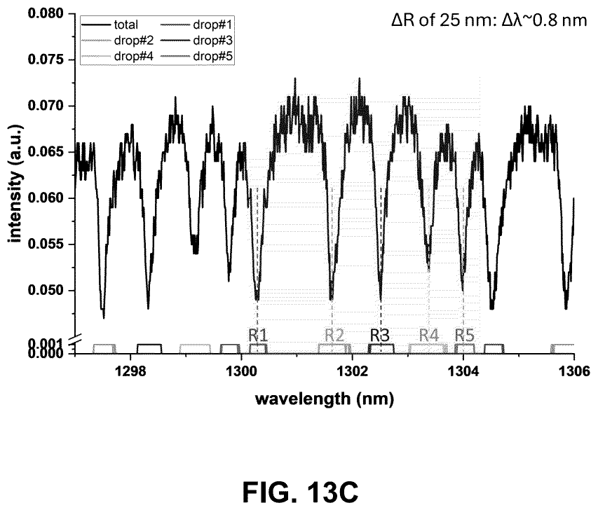

FIG. 13A provides a SEM image of 1 by 5 MRR array with drop lines. The transmission spectrum of the MMR array was measured with drop lines by using the tunable laser and photodetector through each output drop line #1-#5 and a total output port. FIG. 13B provides a SEM image of enlarged view of one MMR unit comprising of a bus waveguide for input and output, a ring resonator, and a drop line. FIG. 13C provides a measured resonance spectrum from the total output port and drop lines. The result shows resonance wavelengths in total spectrum match the output through the drop lines.

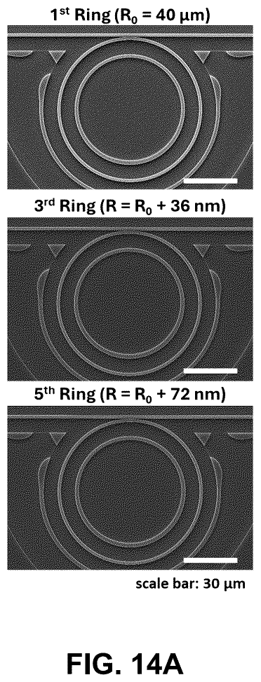

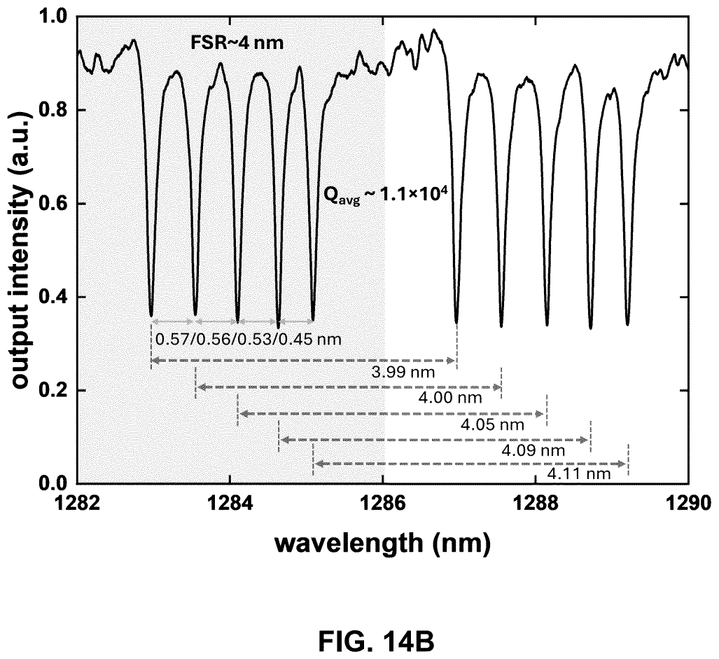

FIG. 14A provides a SEM image of first, third, and fifth ring resonator of 1 by 5 MRR array with an initial radius of 40 μm and an incremental radius of 18 nm. FIG. 14B provides a measured resonance spectrum of the 1 by 5 MRR array. The results show clearly resolvable five resonance curves in one free spectral range (FSR). the average Q-factor is 1.1×10⁴.

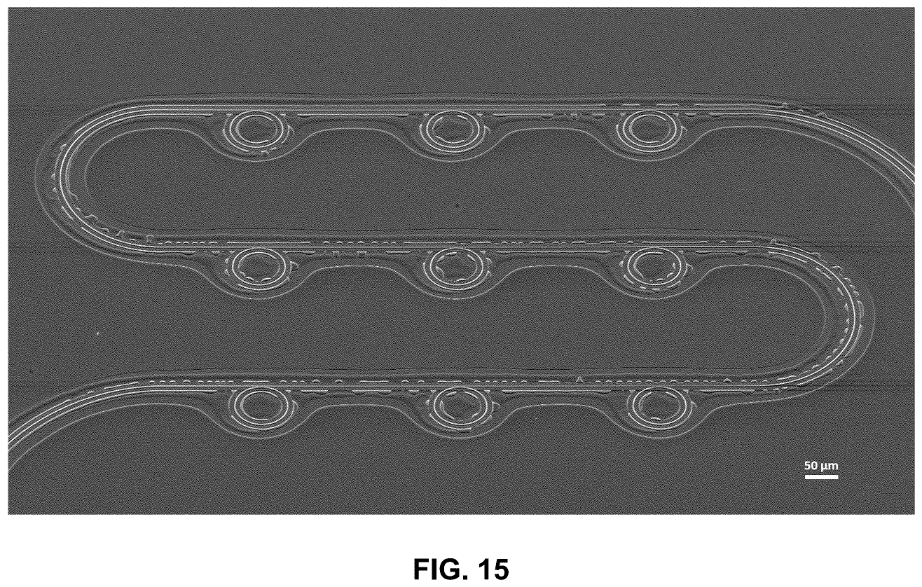

FIG. 15 provides a SEM image of 3 by 3 MRR array with one bus waveguide.

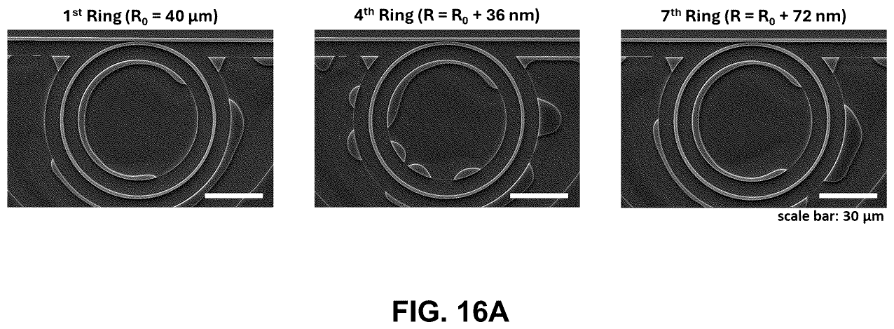

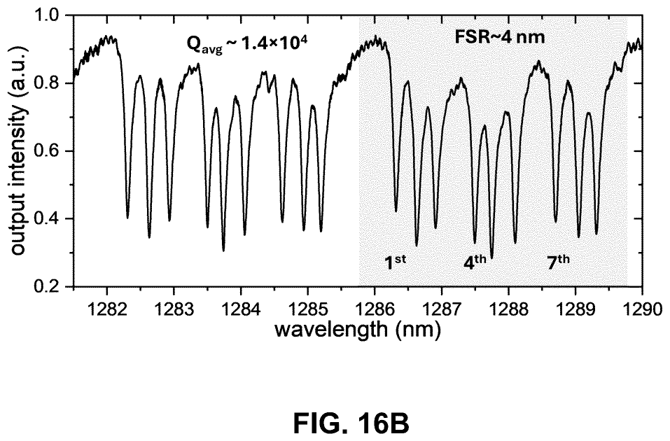

FIG. 16A provides a SEM image of first, fourth, and seventh ring resonator of 3 by 3 MRR array with an initial radius of 40 μm and an incremental radius of 12 nm. FIG. 16B provides measured resonance spectrum of the 3 by 3 MRR array. The result shows the clearly resolvable 9 resonance curves in one free spectral range (FSR). Their average Q-factor is 1.4×10⁴.

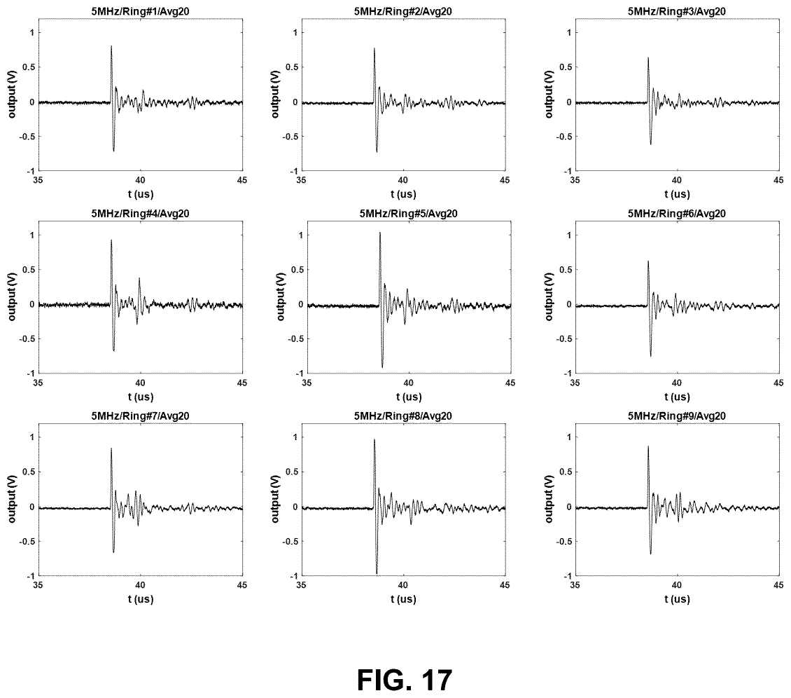

FIG. 17 provides measured ultrasonic signals from each ring of the 3 by 3 MRR array. The input ultrasonic signal has a center frequency of 5 MHz.

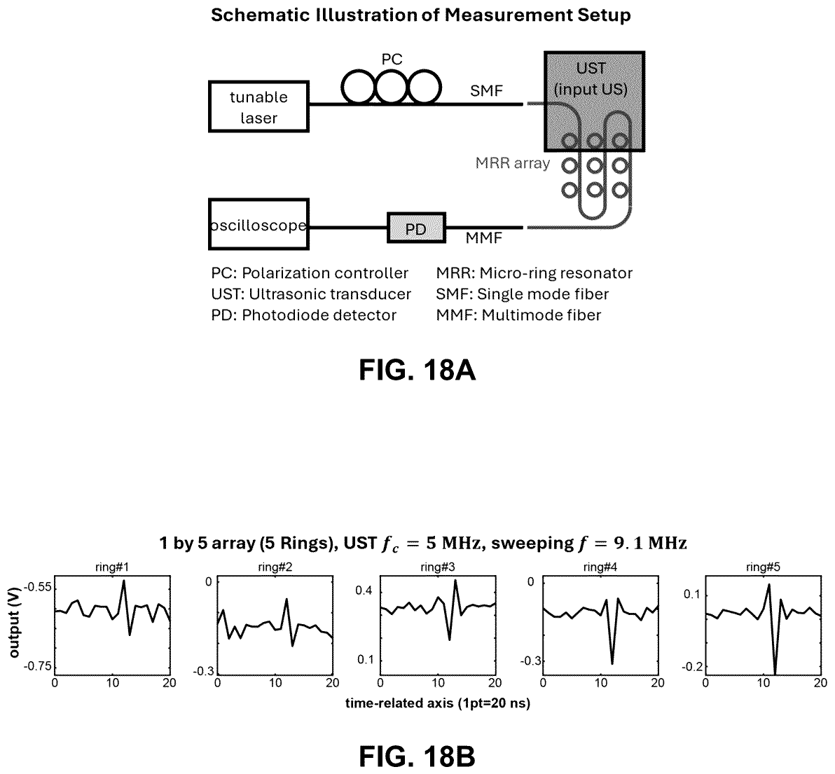

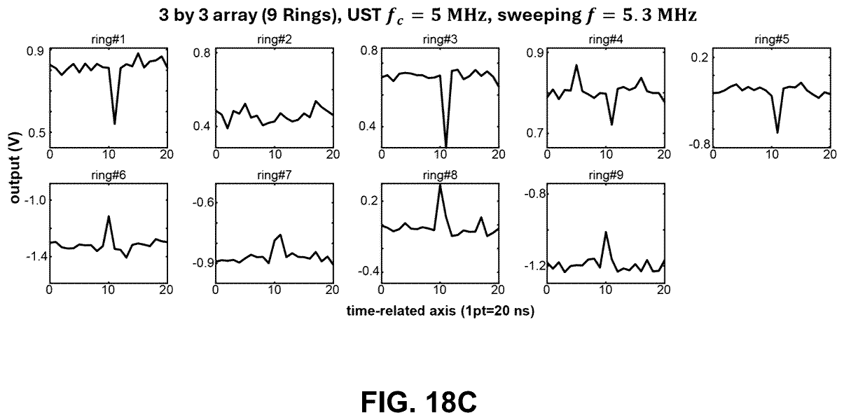

FIG. 18A provides a schematic illustration of parallel detection of ultrasonic signal using the MRR array. FIG. 18B provides the reconstructed ultrasonic signal from each ring of the 1 by 5 MRR array in parallel detection. The input ultrasonic signal has a center frequency of 5 MHz. The wavelength sweeping frequency is 9.1 MHz. FIG. 18C provides the reconstructed ultrasonic signal from each ring of the 3 by 3 MRR array in parallel detection. The input ultrasonic signal has a center frequency of 5 MHz. The wavelength sweeping frequency is 5.3 MHz.

### REFERENCES

1. Li et al. “Disposable ultrasound-sensing chronic cranial window by soft nanoimprinting lithography,” Nature Communications 10, 4277 (2019).
2. Lee et al. “Highly sensitive ultrasound detection using nanofabricated polymer micro-ring resonators,” Nano Convergence 10, 30 (2023).
3. Dong et al. “Isometric multimodal photoacoustic microscopy based on optically transparent micro-ring ultrasonic detection,” Optica 2, 169-176 (2015).
4. Dong et al. “Photoacoustic probe using a micro-ring resonator ultrasonic sensor for endoscopic applications,” Optics Letters 39, 4372-4375 (2014).
5. Pan et al. “Parallel interrogation of the chalcogenide-based micro-ring sensor array for photoacoustic tomography.” Nature Communications 14, 3250 (2023).
6. Westerveld et al. “Sensitive, small, broadband and scalable optomechanical ultrasound sensor in silicon photonics.” Nature Photonics 15, pages 341-345 (2021).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein. One of ordinary skill in the art will appreciate that all art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. An ultrasonic detector for a photoacoustic imaging system, the ultrasonic detector comprising:
a micro-ring resonator array configured to receive light emitted by a driving light source, the micro-ring resonator array comprising a plurality of micro-ring resonators, each respective micro-ring resonator having a respective radius that corresponds to a driving wavelength of the respective micro-ring resonator, wherein the respective radius of each respective micro-ring resonator is different than the respective radii of the other micro-ring resonators.

2. The ultrasonic detector according to claim 1, wherein each micro-ring resonator is configured to deform in the presence of ultrasonic pressure waves emitted from a sample.

3. The ultrasonic detector according to claim 1, further comprising a bus waveguide that connects each respective micro-ring resonator of the plurality of micro-ring resonators in series.

4. The ultrasonic detector according to claim 1, further comprising a plurality of bus waveguides, wherein each respective bus waveguide of the plurality of bus waveguides connects, in series, a respective subset of micro-ring resonators of the plurality of micro-ring resonators, and wherein the plurality of subsets of micro-ring resonators are connected in parallel.

5. The ultrasonic detector according to claim 4, wherein at least one respective bus waveguide of the plurality of bus waveguides includes an optical delay line.

6. An ultrasonic detection system for a photoacoustic imaging system, the ultrasonic detection system including:
the ultrasonic detector of claim 1; and
a driving light source.

7. The ultrasonic detection system according to claim 6, wherein the driving light source is a tunable light source or a broadband laser source comprising a wavelength filter.

8. The ultrasonic detection system according to claim 6, wherein the driving light source is a swept source laser configured to output a laser beam having a wavelength that varies across a range of wavelengths as a function of time; and wherein the range of wavelengths extends from a first wavelength to a second wavelength, wherein the first wavelength is at or below a wavelength that corresponds to a shortest driving wavelength of the plurality of micro-ring resonators, and wherein the second wavelength is at or above a longest driving wavelength of the plurality of micro-ring resonators.

9. The ultrasonic detection system of claim 8, wherein the swept laser source is configured to vary the wavelength of the laser beam between respective driving wavelengths of the plurality of micro-ring resonators, wherein the wavelength of the laser beam is varied such that it occupies one or more off-resonance wavelengths between different respective driving wavelengths.

10. The ultrasonic detector according to claim 8, wherein the swept laser source is a pulsed laser source configured to emit a series of pulses, wherein every other pulse has a wavelength that corresponds to a driving wavelength of the plurality of micro-ring resonators; and wherein the swept laser source is configured to emit, between pulses that correspond to a driving wavelength of the plurality of micro-ring resonators, a pulse having a wavelength that is equal to one or more off-resonance wavelengths.

11. The ultrasonic detection system according to claim 6, further comprising:
a photodetector configured to convert optical signals output through the micro-ring resonator array into electrical signals; and
processing circuitry configured to receive the electrical signals and reconstruct two-dimensional and/or three-dimensional images of a sample.

12. The ultrasonic detection system according to claim 11, wherein the processing circuitry is configured to use electrical signals corresponding to optical signals output by the micro-ring resonator array at points in time at which the wavelength of the laser beam occupies one of the one or more off-resonance wavelengths as a clock signal.

13. The ultrasonic detection system according to claim 11, wherein the optical signals output through the micro-ring resonator array are modulated by photoacoustic signals emitted by a sample, and
wherein the photodetector converts the modulation in the optical signals into the electrical signals.

14. The ultrasonic detection system according to claim 11, wherein the optical signals output through the micro-ring resonator are a time-sequence of pulsed optical signals that exhibit a time-sequence of modulation with alternating high and low optical power, and
wherein the photodetector converts the time-sequence of pulsed optical signals into the electrical signals,
wherein the electrical signals are time-modulated pulsed electric signals that are used as an internal clock, by the processing circuitry, to accurately assign a component of the electrical signals to a deformation of a corresponding micro-ring resonator to the micro-ring resonator array.

15. The ultrasonic detection system according to claim 13, wherein the processing circuitry is further configured to detect phase variations in the optical signals output through the micro-ring resonator array, wherein the phase variations result from deformations caused by the presence of ultrasonic pressure waves emitted from a sample.

16. The ultrasonic detection system according to claim 11, wherein the photodetector comprises a first photodetector port configured to receive the optical signals output by the micro-ring resonator array, and
wherein the ultrasonic detector further comprises a coupler waveguide configured to receive the light emitted by the driving light source and to guide the light emitted by the driving light source to a second photodetector port.

17. The ultrasonic detection system according to claim 16, wherein the photodetector is a balanced photodetector including the second photodetector port, the photodetector being further configured to output second electrical signals, or the ultrasonic detector further comprises a second photodetector comprising the second photodetector port, the second photodetector being configured to output second electrical signals,
wherein the second electrical signals correspond to second optical signals provided to the second photodetector port, and
wherein the balanced photodetector increases a signal-to-noise ratio to enhance detection sensitivity; and
further comprising processing circuitry configured to receive the electrical signals and the second electrical signals and to detect variations in the respective radius of each respective micro-ring resonator resulting from deformations caused by the presence of ultrasonic pressure wave emitted from the sample.

18. A photoacoustic imaging system, comprising:
a pulsed laser configured to illuminate a sample with laser pulses; and
the ultrasonic detection system according to claim 6, wherein the ultrasonic detector is configured to detect ultrasonic pressure waves emitted from the sample.

19. A method for photoacoustic imaging of a sample, the method comprising:
providing an ultrasonic detector according to claim 1;
detecting, by the ultrasonic detector, ultrasonic pressure waves emitted from a sample; and
processing, by processing circuitry, electrical signals corresponding to the optical signals output through the micro-ring resonator array to reconstruct a two-dimensional and/or a three-dimensional image of the sample.

20. The method according to claim 19, wherein the ultrasonic detector is configured to detect the ultrasonic pressure waves via: intensity-based detection; or phase-based detection.
